(19) United States
(12) Patent Application Publication
(10) Pub. No.: US 2010/0004638 A1
(43) Pub. Date: Jan. 7, 2010

(54) Implantable Infusion Devices With Overfill Protection

(76) Inventor: Scott R. Gibson, Granada Hills, CA (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/166,240

(22) Filed: Jul. 1, 2008

(51) Int. Cl. A61K 9/22

(52) U.S. Cl. 604/891.1

(57) ABSTRACT

Implantable medical devices that prevent over fill.

### BACKGROUND OF THE INVENTIONS

1. Field of Inventions

The present inventions relate generally to implantable infusion devices.

2. Description of the Related Art

Implantable infusion devices have been used to provide a patient with a medication or other substance (collectively “infusible substance”) and frequently include a reservoir and a pump. The reservoir is used to store the infusible substance and, in some instances, implantable infusion devices are provided with a fill port that allows the reservoir to be transcutaneously filled (and/or re-filled) with a hypodermic needle. The reservoir is coupled to the pump, which is in turn connected to an outlet port. A catheter, which has at least one outlet at the target body region, may be connected to the outlet port. As such, infusible substance may be transferred from the reservoir to the target body region by way of the pump and catheter.

One issue associated with implantable infusion devices is “overfill.” Overfill, which occurs when a clinician supplies more infusible substance to the fill port than the implantable infusion device can accommodate, may lead to damage to the device and/or harm to the patient. The present inventor has determined that conventional overfill protection apparatus are susceptible to improvement. For example, some overfill protection apparatus employ an inlet valve that is connected to, or is otherwise configured to remain in contact with, a movable portion of the reservoir. The inlet valve has a valve element that moves to its closed position when the reservoir movable portion reaches the position associated with a full reservoir, thereby preventing any additional flow through the valve. U.S. Pat. No. 5,158,547 to Doan et al. discloses such a valve. The present inventor has determined that such valves undesirably increase the thickness of the associated implantable infusion device because there is a fixed length connector between the movable portion of the reservoir and the valve element, and space must provided to accommodate the fixed length connector when the reservoir is empty.

### SUMMARY OF THE INVENTIONS

An implantable medical device in accordance with one embodiment of a present invention includes a reservoir with a movable member, a fill port, and a valve which has a valve element and a variable length connector that operably connects the valve element to the reservoir movable member. The variable length connector may, for example, be longer when the reservoir is full than when the reservoir is empty. As such, less space is required to accommodate the connector when the reservoir is empty.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a plan view of an implantable infusion device in accordance with one embodiment of a present invention.

FIG. 2 is a plan view of the exemplary implantable infusion device illustrated in FIG. 1 with the cover removed.

FIG. 3 is a partial section view taken along line 3-3 in FIG. 1.

FIG. 4 is a block diagram of the exemplary implantable infusion device illustrated in FIGS. 1-3.

FIG. 5 is a section view of a portion of the exemplary implantable infusion device illustrated in FIG. 1 with the inlet valve open.

FIG. 6 is an enlarged view of a portion of FIG. 5.

FIG. 7 is a bottom plan view of a needle stop in accordance with one embodiment of a present invention.

FIG. 8 is a bottom plan view of a valve seat in accordance with one embodiment of a present invention.

FIG. 9 is a top plan view of a valve seat in accordance with one embodiment of a present invention.

FIG. 10 is a section view taken along line 10-10 in FIG. 8.

FIG. 11 is a top view of a linkage cap in accordance with one embodiment of a present invention.

FIG. 12 is a section view taken along line 12-12 in FIG. 11.

FIG. 13 is a section view taken along line 13-13 in FIG. 12.

FIG. 14 is a section of a portion of the exemplary implantable infusion device illustrated in FIG. 1 with the inlet valve closed.

FIG. 15 is an enlarged view of a portion of FIG. 14.

FIG. 16 is a section view of a variable length connector in accordance with one embodiment of a present invention.

FIG. 17 is a section view of a variable length connector in accordance with one embodiment of a present invention.

FIG. 18 is a section view of a variable length connector in accordance with one embodiment of a present invention.

FIG. 19 is a section view of a portion of a closed valve in accordance with one embodiment of a present invention.

FIG. 20 is a plan view of a valve element in accordance with one embodiment of a present invention.

FIG. 21 is a section view of a portion of an open valve in accordance with one embodiment of a present invention.

FIG. 22 is a plan view of an implantable infusion device system in accordance with one embodiment of a present invention.

FIG. 23 is a block diagram of the implantable infusion device system illustrated in FIG. 22.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions are also not limited to the exemplary implantable infusion devices described herein and, instead, are applicable to other implantable infusion devices that currently exist or are yet to be developed.

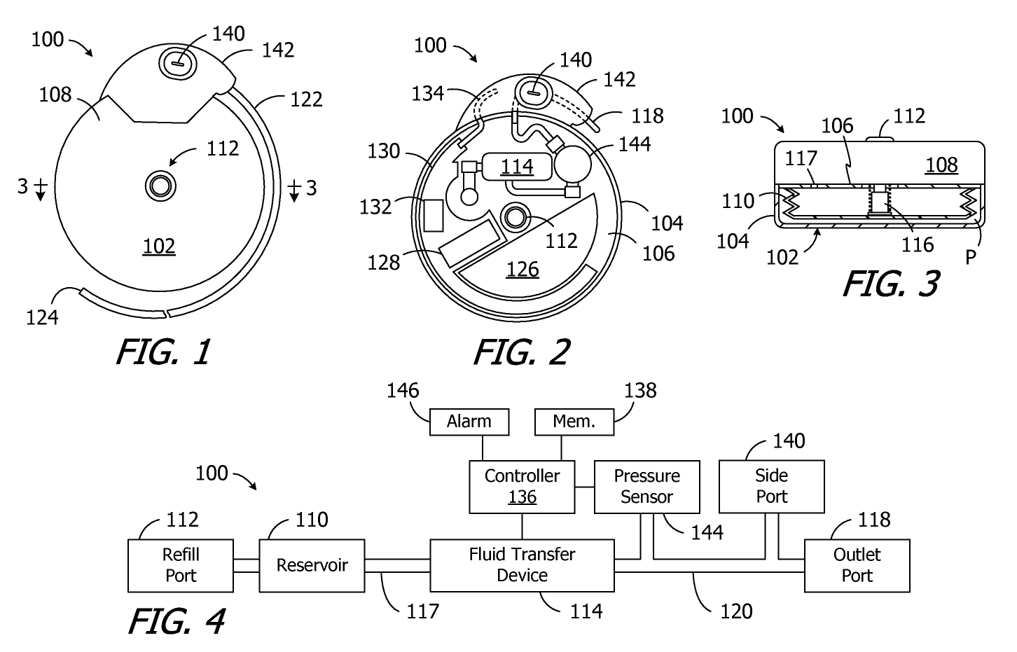

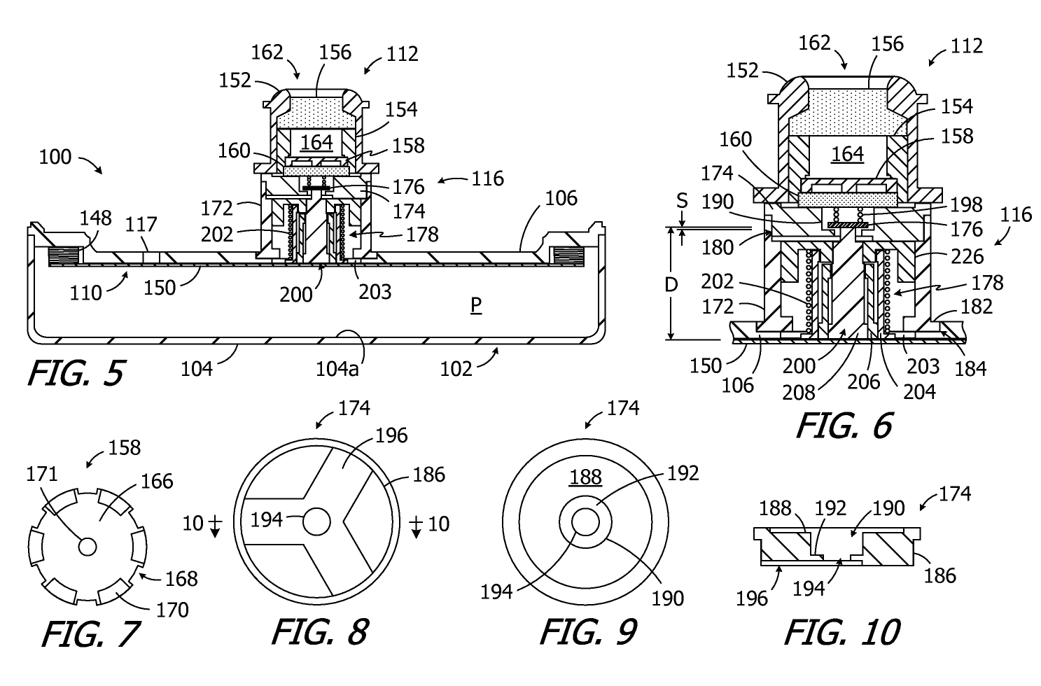

One example of an implantable infusion device in accordance with a present invention is generally represented by reference numeral 100 in FIGS. 1-4. As used herein, an “implantable infusion device” is a device that includes a reservoir and an outlet, and is sized, shaped and otherwise constructed (e.g. sealed) such that both the reservoir and outlet can be simultaneously carried within the patient's body. The exemplary infusion device 100 includes a housing 102 (e.g. a titanium housing) with a bottom portion 104, an internal wall 106, and a cover 108. An infusible substance (e.g. medication) may be stored in a reservoir 110 that is located within the housing bottom portion 104. The reservoir 110 may be replenished by way of a fill port 112 that extends from the reservoir to the cover 108. A hypodermic needle (not shown), which is configured to be pushed through the fill port 112, may be used to replenish the reservoir 110. Fluid flow from the fill port 112 to the reservoir 110 is controlled by an inlet valve 116. The reservoir 110, fill port 112 and inlet valve 116 are discussed in greater detail below with reference to FIGS. 5-21.

A wide variety of reservoirs may be employed. In the illustrated embodiment, the reservoir 110 is in the form of a titanium bellows that is positioned within a sealed volume defined by the housing bottom portion 104 and internal wall 106. The remainder of the sealed volume is occupied by propellant P, which may be used to exert negative pressure on the reservoir 110. Other reservoirs that may be employed in the present infusion devices include reservoirs in which propellant exerts a positive pressure. Still other exemplary reservoirs include negative pressure reservoirs that employ a movable wall that is exposed to ambient pressure and is configured to exert a force that produces an interior pressure that is always negative with respect to the ambient pressure.

The exemplary ambulatory infusion device 100 illustrated in FIGS. 1-4 also includes a fluid transfer device 114. The inlet of the fluid transfer device 114 is coupled to the interior of the reservoir 110 by a passageway 117, while the outlet of the fluid transfer device is coupled to an outlet port 118 by a passageway 120. Operation of the fluid transfer device 114 causes infusible substance to move from the reservoir 110 to the outlet port 118. A catheter 122 may be connected to the outlet port 118 so that the infusible substance passing through the outlet port will be delivered to a target body region in spaced relation to the infusion device 100 by way of the outlet 124 at the end of the catheter.

A wide variety of fluid transfer devices may be employed. In the illustrated embodiment, the fluid transfer device 114 is in the form of an electromagnet pump. The present inventions are not, however, limited to electromagnet pumps and may include other types of fluid transfer devices. Such devices include, but are not limited to, other electromagnetic pumps, solenoid pumps, piezo pumps, and any other mechanical or electromechanical pulsatile pump. Additionally, in the context of positive pressure reservoirs, the fluid transfer device may be in the form of an accumulator which includes a variable volume housing and active inlet and outlet valves. In the exemplary context of implantable drug delivery devices, and although the volume/stroke magnitude may be increased in certain situations, the fluid transfer devices will typically deliver about 1 microliter/stroke or other actuation, but may be more or less (e.g. about 0.25 microliter/actuation or less) depending on the particular fluid transfer device employed. Additionally, although the exemplary fluid transfer device 114 is provided with internal valves (e.g. a main check valve and a bypass valve), valves may also be provided as separate structural elements that are positioned upstream of and/or downstream from the associated fluid transfer device.

Energy for the fluid transfer device 114, as well for other aspects of the exemplary infusion device 100, is provided by the battery 126 illustrated in FIG. 2. In the specific case of the fluid transfer device 114, the battery 126 is used to charge one or more capacitors 128, and is not directly connected to the fluid transfer device itself. The capacitor(s) 128 are connected to an electromagnet coil in the fluid transfer device 114, and disconnected from the battery 126, when the electromagnet coil is being energized, and are disconnected from the electromagnet coil and connected to the battery when the capacitor(s) are being recharged and/or when the fluid transfer device is at rest. The capacitor(s) 128 are carried on a board 130. A communication device 132, which is connected to an antenna 134, is carried on the same side of the board 130 as the capacitor(s) 128. The exemplary communication device 132 is an RF communication device. Other suitable communication devices include, but are not limited to, oscillating magnetic field communication devices, static magnetic field communication devices, optical communication devices, ultrasound communication devices and direct electrical communication devices.

A controller 136 (FIG. 4), such as a microprocessor, microcontroller or other control circuitry, is carried on the other side of the board 130. The controller controls the operations of the infusion device 100 in accordance with instructions stored in memory 138 and/or provided by an external device by way of the communication device 132. For example, the controller 136 may be used to control the fluid transfer device 114 to supply fluid to the patient in accordance with, for example, a stored basal delivery schedule or a bolus delivery request. The controller 136 may also be used to monitor sensed pressure and perform the analytical functions described below.

Referring to FIGS. 1, 2 and 4, the exemplary infusion device 100 is also provided with a side port 140 that is connected to the passageway 120 between the outlet of the fluid transfer device 114 and the outlet port 118. The side port 140 facilitates access to an implanted catheter 122, typically by way of a hypodermic needle. For example, the side port 140 allows clinicians to push fluid into the catheter 122 and/or draw fluid from the catheter for purposes such as checking catheter patency, sampling CSF, injecting contrast dye into the patient and/or catheter, removing medication from the catheter prior to dye injection, injecting additional medication into the region at the catheter outlet 124, and/or removing pharmaceuticals or other fluids that are causing an allergic or otherwise undesirable biologic reaction.

The outlet port 118, a portion of the passageway 120, the antenna 134 and the side port 140 are carried by a header assembly 142. The header assembly 142 is a molded, plastic structure that is secured to the housing 102. The housing 102 includes a small aperture through which portions of the passageway 120 are connected to one another, and a small aperture through which the antenna 134 is connected to the board 130.

The exemplary infusion device 100 illustrated in FIGS. 1-4 also includes a pressure sensor 144 that is connected to the passageway 120 between the outlet of the fluid transfer device 114 and the outlet port 118. As such, the pressure sensor 144 senses the pressure at the outlet port 118 which, in the illustrated embodiment, is also the pressure within the catheter 122. The pressure sensor 144 is connected to the controller 136 and may be used to analyze a variety of aspects of the operation of the exemplary implantable infusion device 100. For example, pressure measurements may be used by the controller 136 to determine whether or not there is a blockage in the catheter 122 and whether or not the fluid transfer device 114 is functioning properly. The controller 136 may perform a variety of different functions in response to a determination that the fluid transfer device 114 is not functioning properly or a determination that the catheter 122 is blocked. For example, the controller 136 may actuate an audible alarm 146 that is located within the housing 102 in order to signal that the fluid transfer device 114 is not functioning properly or the catheter 122 is blocked.

Turing to FIGS. 5 and 14, and as noted above, the exemplary reservoir 110 is a titanium bellows. The titanium bellows includes a plurality of convolutes 148 and a terminal diaphragm 150. The convolutes 148 expand as the reservoir 110 moves from the empty orientation (FIG. 5) to the full orientation (FIG. 14).

As illustrated for example in FIGS. 5, 6, 14 and 15, the fill port 112 in the illustrated embodiment has a housing 152, a base 154, a septum 156, a needle stop 158 and a filter 160. The fill port housing 152 includes an internal lumen 162 through which a needle may be inserted. The base 154, which supports the septum 156 and centers the needle stop 158 and filter 160, includes an internal lumen 164 through which a needle may be inserted. The septum 156 may, for example, be a self-healing septum formed from materials such as silicone. The needle stop 158 limits needle travel while allowing fluid flow and, in the illustrated embodiment, includes a stop member 166 with a plurality of apertures 168, a plurality of perimeter supports 170, and a center support 171 (FIG. 7). The supports 170 and 171 create spacing between the stop member 166 and the filter 160 to facilitate fluid flow from the apertures 168 to the entire top surface of the filter. Although other types of filters may be employed, the filter 160 in the illustrated embodiment is a sintered metallic filter.

The exemplary inlet valve 116 illustrated in FIGS. 5, 6, 14 and 15 includes a valve housing 172, a valve seat 174, a valve element 176, and a variable length connector 178 that operably connects the valve element to the reservoir terminal diaphragm 150. Although not limited to any particular shape, the exemplary valve housing 172 has a generally cylindrical shape and includes a recess 180 for the valve seat 174 and a base 182 that is received within a recess 184 in the internal wall 106 (6 and 15).

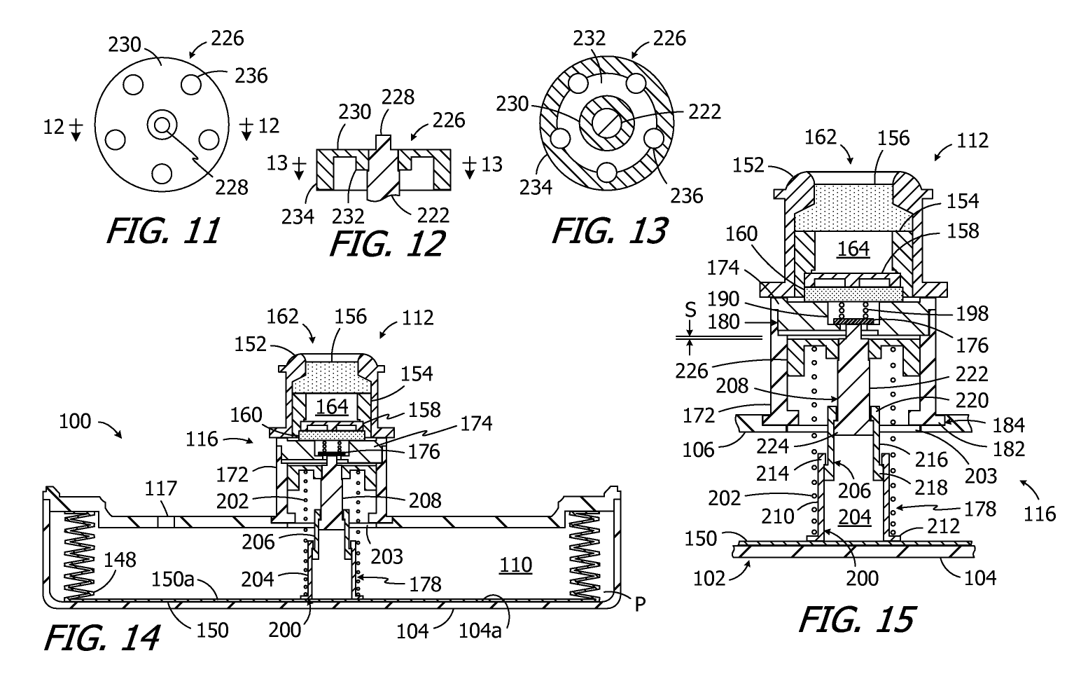

Turning to FIGS. 8-10, the exemplary valve seat 174 includes a narrow portion 186 that is received within the valve housing recess 180, an indentation 188 in the top surface (in the illustrated orientation) for the filter 160, a lumen 190 in which the valve element 176 is located, a seal surface 192, and an aperture 194 through which fluid flows when the valve element is in the open position in spaced relation to the seal surface (FIG. 6). The bottom surface (in the illustrated orientation) of the exemplary valve seat 174 includes a plurality of fluid channels 196. The fluid channels 196 connect the aperture 194 to the lumens 236 in the connector cap 226 (discussed below with reference to FIGS. 11-13).

The exemplary valve element 176 illustrated in FIGS. 5, 6, 14 and 15 is an elastomeric disk which, as noted above, is located within the valve seat lumen 190. In the illustrated embodiment, the valve element 176 is biased to the closed position (FIGS. 14 and 15) by a biasing device 198, such as a spring (e.g. a coil spring) that is compressed between the filter 160 and the valve element. In other embodiments, the biasing device 198 may be omitted. The valve element 176 may, in some embodiments, be configured to reduce the likelihood that it will stick to the valve seat seal surface 192. To that end, the valve element 176 may be formed from an elastomer (e.g. silicone rubber) and may include a non-stick surface (not shown) on the side of the valve element that abuts the valve seat seal surface 192. The non-stick surface, which may be in the form of a layer of silicon suboxide (SiOₓC_{y}, where x<2 and y<1) such as, for example, SiO_{1.7}C_{0.4}, is relatively thin and plasma deposition (or other suitable techniques) may be used to deposit the silicon suboxide onto the valve element 176. The thickness of a relatively thin layer of silicon suboxide is about 0.1 μm to about 10 μm and, in the context of the illustrated embodiment, may be about 0.3 μm to about 0.8 μm thick. The non-stick surface may also cover the entire bottom surface (in the illustrated orientation) of the valve element 176, only that part that would engage the seal surface 192, or something in between. The non-stick surface may, alternatively, be a layer of SiO₂ that is about 0.1 μm to about 10 μm thick and formed by an oxygenated plasma treatment of the valve element 176. It should also be noted here that, in other implementations, the valve element 176 may be formed from metal or hard plastic and the valve seat seal surface 192 may be formed from an elostomer, with or without the non-stick surface. Still another alternative is to form the valve element 176 and the valve seat seal surface 192 from metal or hard plastic, so long as the resulting flow restriction is sufficient to provide a perceptible indication of flow cessation.

Referring now to FIGS. 6 and 15, the exemplary variable length connector 178 abuts, but is not secured to, the reservoir terminal diaphragm 150 and the valve element 176 in the illustrated embodiment. In other embodiments, however, the variable length connector may be secured to the reservoir terminal diaphragm 150 and/or the valve element 176. The exemplary variable length connector 178 includes an expandable structure 200 and a biasing element 202, such as a coil spring, that biases the expandable structure to the fully extended orientation illustrated in FIG. 15.

The configuration of the housing 102 (e.g., the distance between the inner surfaces of the bottom portion 104 and internal wall 106) and/or the configuration of the reservoir 110, and the configuration of the variable length connector 178 are such that the variable length connector will hold the valve element 176 in the open position (FIGS. 5 and 6) until the reservoir is filled and the terminal diaphragm 150 reaches the position illustrated in FIGS. 14 and 15. In particular, as the reservoir 110 is being filled and the terminal diaphragm 150 is moving from the position illustrated in FIGS. 5 and 6 to the position illustrated in FIGS. 14 and 15, the length of the variable length connector 178 will increase from its smallest length to its maximum length. The variable length connector 178 will not, when terminal diaphragm 150 reaches the position illustrated in FIGS. 14 and 15, hold the valve element 176 in the open position. The maximum length of the variable length connector is less than the distance D (FIG. 6) between the bottom surface of the valve element 176, in its open position, and the inner surface of the terminal diaphragm 150 when the reservoir is full. The valve element 176 will then move, due to the force exerted by the biasing element 198 and/or the pressure differential across the valve element, to the closed position (FIGS. 14 and 15).

The exemplary expandable structure 200 is configured such that, when it is in the fully compressed stated illustrated in FIGS. 5 and 6, it will be almost entirely located above the internal wall 106 in the pump housing 102. The internal wall 106 is provided with an opening 203 (FIGS. 5, 6, 14 and 15) through which the expandable structure 200 and biasing element 202 extend when the length of the variable length connector 178 is greater than that illustrated in FIGS. 5 and 6. Fluid from the fill port 112 also passes through the opening on its way to the reservoir 110 when the inlet valve 116 is open.

A wide variety of variable length connectors may be employed. Referring to FIG. 15, the exemplary expandable structure 200 in the exemplary variable length connector 178 is a telescoping structure that includes an outer member 204, a middle member 206, and an inner member 208. The outer member 204 has a hollow tubular body 210, an outwardly extending flange 212 that abuts the reservoir terminal diaphragm 150 and biasing element 202, and an inwardly extending flange 214. The middle member 206 has a hollow tubular body 216, an outwardly extending flange 218 that abuts the outer member inwardly extending flange 214 when the expandable structure is fully expanded, and an inwardly extending flange 220. The inner member 208 has an elongate body 222 that may be solid (as shown) or hollow, an outwardly extending flange 224 that abuts the middle member inwardly extending flange 220 when the expandable structure is fully expanded, a cap 226 that is configured to permit fluid to flow therethrough and to engage one end of the biasing element 202, and a protrusion 228 (FIG. 12) that extends beyond the cap. The protrusion 228 is the portion of the expandable structure 200 that engages the valve element 176. The tubular and elongate bodies may be circular in cross-section, i.e., cylindrical, as shown or may have other cross-sectional shapes.

Referring to FIGS. 11-13, the exemplary cap 226 includes a disk 230, an inner member 232 and an outer member 234. The disk 230 faces the valve seat 174, the inner member 232 is secured to the elongate body 222 (e.g. through the use of an interference fit), and the outer member 234 faces the inner surface of the valve housing 172. A plurality of fluid lumens 236 extend though the disk 230 and the outer member 234. The respective size, spacing and number of the cap lumens 236 and the valve seat fluid channels 196 (FIG. 8) are such that, no matter how the cap 226 is rotationally oriented relative to the valve seat 174, fluid that flows through the valve seat aperture 194 will also flow through at least some of the cap lumens 236 and into the reservoir 110 by way of the opening 203.

With respect to operation, the valve 116 is configured to remain open when the reservoir is not full and to close when the reservoir 110 is full. More specifically, and although the process may begin when the reservoir 110 is partially full, the reservoir is empty in FIG. 5 and 6. The exemplary variable length connector 178 is in its shortest state (and the expandable structure 200 is fully compressed) due to the force exerted on the variable length connector by the terminal diaphragm 150, and the valve 116 is in its open state. The cap 226 is urged against the valve seat 174 by the biasing element 202 and, accordingly, the inner member protrusion 228 maintains a space S between the valve element 176 and the seal surface 192 (FIG. 10). The space S is sized such that there will be little resistance to flow, both into and out of the reservoir 110, and the space S is about 0.003 inches in the illustrated implementation.

The terminal diaphragm 150 will move toward the end wall 104a of the housing bottom portion 104, and the length of the variable length connector 178 will increase, as infusible substance enters the reservoir 110 by way of the fill port 112, open valve 116 and opening 203. In particular, the force exerted on the outer member 204 and cap 226 by the biasing element 202 will cause the expandable structure 200 to expand as the terminal diaphragm 150 moves. The middle member 206 is, at this point, free to move (or “float”) relative to the outer member 204 and inner member 208. The cap 226 will continue to be urged against the valve seat 174 by the biasing element 202 and, accordingly, the inner member protrusion 228 will continue to maintain a space S between the valve element 176 and the seal surface 192, and keep the valve 116 open, by virtue of the force applied to the variable length connector 178 by the reservoir terminal diaphragm 150.

Turning to FIGS. 14 and 15, in the exemplary embodiment, the terminal diaphragm 150 will abut the end wall 104a of the housing bottom portion 104 when the reservoir 110 is full. At this point, the force exerted on the outer member 204 and cap 226 by the biasing element 202 will cause the expandable structure 200 to be in its fully expanded state and, accordingly, the variable length connector 178 will be at its maximum length. The outwardly and inwardly extending flanges 218 and 220 of the middle member 206 will be forced into contact with the inwardly extending flange 214 of the outer member 204 and the outwardly extending flange 224 of the inner member 208. When the variable length connector 178 is at its maximum length, the distance between the bottom surface of the outer member outwardly extending flange 212 and the top surface of the cap disk 230 (in the illustrated orientation) is slightly less than the distance between the inner surface 150a of the terminal diaphragm 150 and the bottom surface of the valve seat 174. The difference is equal to the length of the space S in the illustrated embodiment. The variable length connector 178 will, therefore, be free to move a short distance relative to the valve element housing 172 and the valve seat 174. In other words, the terminal diaphragm 150 will no longer be exerting an opening force on the valve element 176 by way of the variable length connector 178. The force associated with the biasing device 198 and/or the pressure differential across the valve element 176 will then move the valve element 176 and variable length connector 178 the length of the space S, thereby bringing the valve element into contact with the valve seat seal surface 192 (FIGS. 9 and 10) and closing the valve 116.

The exemplary valve 116 is also configured to reopen as the operation of the fluid transfer device 114 reduces the volume of infusible substance within the reservoir 110 and the terminal diaphragm 150 moves towards the housing wall 106. For example, in the illustrated embodiment, once the volume of the infusible substance in the reservoir 110 has been reduced to the point at which the reservoir is about 95-97% full, the corresponding movement of the terminal diaphragm 150 will have compressed the biasing element 202 to the point at which any further movement of the terminal diaphragm will result in movement of the variable length connector 178 toward to the valve seat 174. The variable length connector 178 will then move the valve element 176 away from the valve seat seal surface 192 to open the valve 116. This aspect of the exemplary embodiment may be adjusted by, for example, adjusting the properties of the biasing element 202. After the cap 226 engages the valve seat 174, additional movement of the terminal diaphragm will simply reduce the length of the expandable structure 200 and compress the biasing element 202.

It should be noted here that a wide variety of variable length connectors may be employed and that the present inventions are not limited to any particular type. By way of example, but not limitation, the variable length connector 178 may be modified by simply increasing or decreasing the number of members in the expandable structure 200.

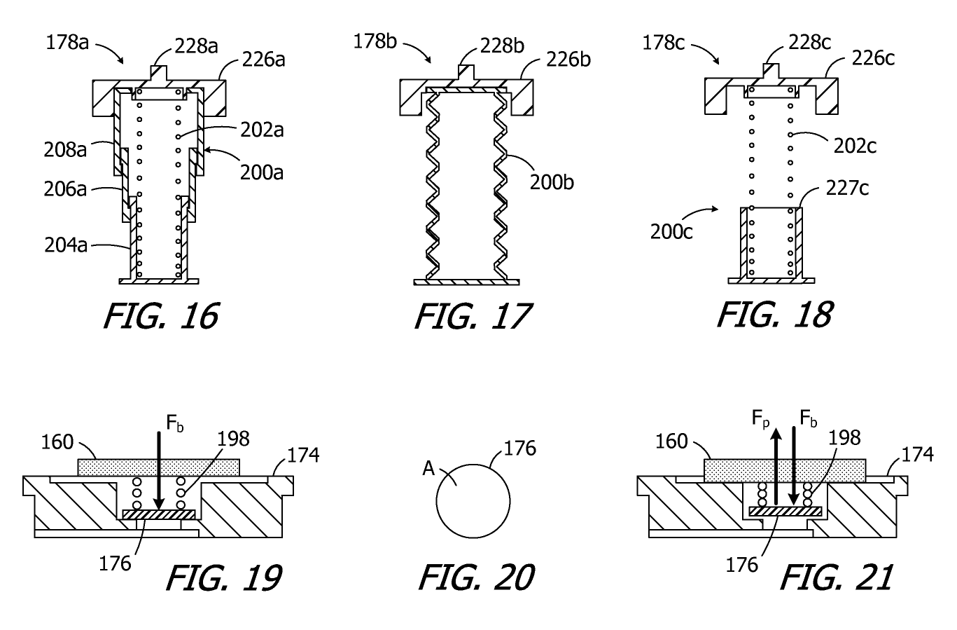

Alternatively, or in addition, a variable length connector may be configured such that biasing device is located inside an expandable structure. One example of such a variable length connector is generally represented by reference numeral 178a in FIG. 16. The variable length connector 178a includes an expandable structure 200a and a biasing element 202a, such as a coil spring, located therein. The exemplary expandable structure 200a is a telescoping structure that includes an outer member 204a, a middle member 206a, and an inner member 208a that are connected to one another in a manner similar to that described above with reference to the expandable structure 200. The outer member 208a also includes a cap 226a. The cap 226a includes lumens (not shown) that are located outwardly of the expandable structure 200a. Alternatively, the cap and expandable structure may be configured such that fluid flows through the cap and the expandable structure. The cap 226a also includes a protrusion 228a.

Another exemplary variable length connector is generally represented by reference numeral 178b in FIG. 17. Here, the expandable structure 200b is in the form of an aneroid, such as a sealed titanium bellows with a compressible gas therein. The expandable structure 200b, which is shown in its neutral state in FIG. 17, includes a cap 226b. The cap 226b has lumens (not shown) that are located outwardly of the expandable structure 200b and a protrusion 228b.

Still another exemplary variable length connector is generally represented by reference numeral 178c in FIG. 18. Here, the expandable structure 200c simply includes a pair of caps 226c and 227c that are carried on opposite ends of a biasing element 202c, such as a coil spring. The cap 226c has lumens (not shown) and a protrusion 228c.

In accordance with another aspect of the illustrated embodiment, the valve element 176 and biasing element 198 may be configured such that the valve 116 will open when the clinician pulls back on a syringe that has been inserted into the fill port 112. More specifically, and referring to FIGS. 19-21, pulling back on a syringe that has been inserted through the septum 156 will create a vacuum and a differential pressure across the valve element 176. The differential pressure and the area A of the valve element 176 results in a force Fₚ being applied to the valve element. The preload force F_{b} applied by the biasing element 198 and the cross-sectional area of the valve element 176 are such that the force Fₚ associated with the differential pressure will overcome the preload force F_{b} associated with the biasing element and move the valve element to the position illustrated in FIG. 21, thereby opening the valve 116. The ability to open the valve 116 in this manner facilitates the removal of fluid from the implantable medical device 100 by way of the fill port 112 if, for example, there is a procedural necessity to do so, such as the realization that the wrong infusible substance has been transferred to the device 100 by way of a syringe. In one exemplary implementation, the surface area of the valve is 0.025 in.² and the preload force F_{b} associated with the biasing element is about 0.02 lb., then differential pressure created by the vacuum would need to be about 0.75 psi (or about 0.5 psi to 2.0 psi, given design and manufacturing tolerances). In those instances where a negative pressure reservoir is employed (e.g. reservoir 110), the negative pressure will also have to be overcome. If, for example, the negative reservoir pressure is 4 psi, then an additional 4 psi of vacuum pressure would have to be created by the syringe, for a total of 4.75 psi in the example above.

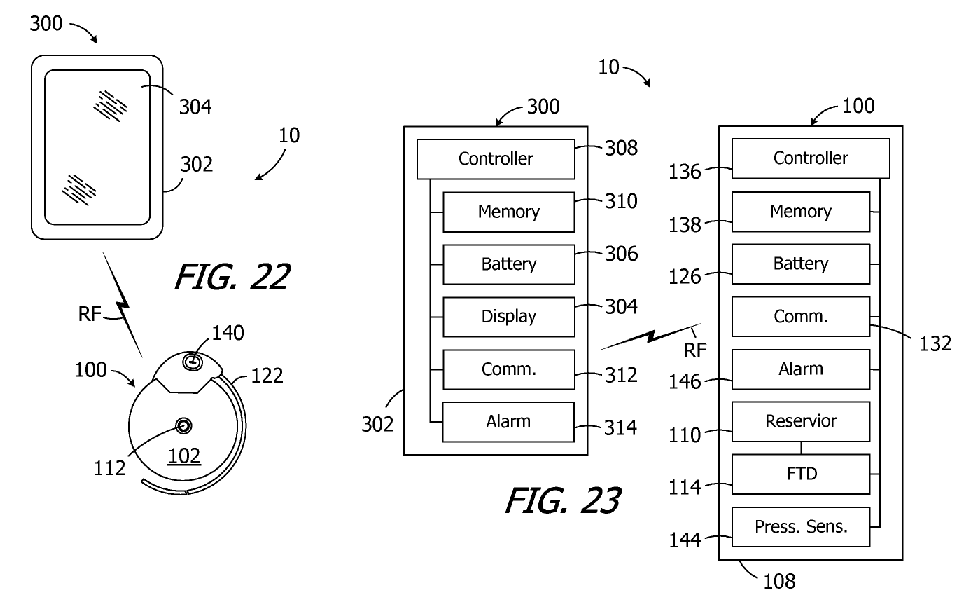

Turning to FIGS. 22 and 23, the exemplary implantable infusion device 100 may be included in an infusion device system 10 that also includes a remote control 300 that is not implanted in the patient. The exemplary remote control 300 includes a housing 302, a touch screen display 304 (or other input device, such as a keypad, with or without a separate display), a battery or other power source 306, a controller 308, such as a microprocessor, microcontroller or other control circuitry, memory 310, and a communication device 312 (including an antenna if necessary). Although the present inventions are not limited to any particular communication device, the exemplary communication device 312 is a telemetry device that transmits an RF signal at a specified frequency. The RF signal may, in some instances, be a carrier signal that carries bit streams. The communication device 312 is configured to send signals to and receive signals from the communication device 132 in the implantable infusion device 100 by way of the antenna 134. Other exemplary communication devices include oscillating magnetic field communication devices, static magnetic field communication devices, optical communication devices, ultrasound communication devices and direct electrical communication devices. In some instances, the remote control may also include an audible alarm 314.

The exemplary remote control 300 may be used to perform a variety of conventional control functions including, but not limited to, turning the infusion device ON or OFF and programming various infusion device parameters. Examples of such parameters include, but are not limited to, the rate of delivery of a given medication, the time at which delivery of a medication is to commence, and the time at which delivery of a medication is to end. Additionally, in at least some implementations, the implantable infusion device 100 will transmit signals to the remote control 300. The signals provide status information about the infusion device 100 that may be stored in memory 310 and/or displayed on the display 304. Examples of such status information include, but are not limited to, the state of charge of the battery 126, the amount of medication remaining in the reservoir 110, the amount of medication that has been delivered during a specified time period, and the presence of a catheter blockage. The signals from the infusion device 100 may also be indicative of sensed physiological parameters in those instances where the infusion device is provided with physiological sensors (not shown).

Although the inventions disclosed herein have been described in terms of the preferred embodiments above, numerous modifications and/or additions to the above-described preferred embodiments would be readily apparent to one skilled in the art. By way of example, but not limitation, the present inventions have application in infusion devices that include multiple reservoirs and/or outlets. It is intended that the scope of the present inventions extend to all such modifications and/or additions and that the scope of the present inventions is limited solely by the claims set forth below.

1. An implantable medical device, comprising:
a reservoir including a movable member that is movable between a reservoir full position and a reservoir empty position;
a fill port in fluid communication with the reservoir;
a valve, between the reservoir and the fill port, which includes
a valve element that is movable between an open position and a closed position and defines first and second sides, and
a variable length connector associated with the first side of the valve element that is not fixedly secured to the valve element, that operably connects the valve element to the reservoir movable member, that is movable between a minimum length and a maximum length, and that is biased by a first bias element to the maximum length; and
a second bias element, which is associated with the second side of the valve element, that biases the valve element to the closed position.

2. An implantable medical device as claimed in claim 1, wherein the reservoir and the variable length connector are respectively constructed and arranged such that the variable length connector and the reservoir movable member hold the valve element in the open position when the reservoir is not full and do not hold the valve element in the open position when the reservoir is full.

3. (canceled)

4. An implantable medical device as claimed in claim 1, wherein the reservoir comprises a bellows reservoir and the movable member comprises a terminal diaphragm.

5. An implantable medical device as claimed in claim 1, wherein the variable length connector is not secured to the reservoir movable member.

6. (canceled)

7. An implantable medical device as claimed in claim 1, wherein the maximum length of the variable length connector is less than or equal to the distance between the valve element and the reservoir movable member when the valve element is in the closed position and the reservoir movable member is in the reservoir full position.

8. An implantable medical device as claimed in claim 1, wherein the variable length connector comprises an expandable structure, including a least two members that are longitudinally movable relative to one another between an expanded state and a compressed state, and the first bias element biases the expandable structure to the expanded state.

9. An implantable medical device as claimed in claim 1, further comprising:
a fluid transfer device operably connected to the reservoir.

10-13. (canceled)

14. An implantable medical device as claimed in claim 1, wherein
the second bias element applies a biasing force to the valve element; and
the biasing force and the configuration of the valve element are such that a differential pressure of about 0.5 psi to about 2.0 psi across the valve element will overcome the biasing force applied by the second bias element.

15. An implantable medical device as claimed in claim 14, wherein the second bias element comprises a spring.

16. An implantable medical device as claimed in claim 15, wherein the fill port includes a filter and the spring is located between the filter and the valve element.

17. An implantable medical device as claimed in claim 14, wherein the fill port includes a septum and the differential pressure is the result of a vacuum force applied between the septum and the valve element.

18. An implantable medical device as claimed in claim 14, wherein the valve element comprises a disk.

19. An implantable medical device as claimed in claim 14, wherein the biasing force and the configuration of the valve element are such that a differential Pressure of about 0.75 psi across the valve element will overcome the biasing force.

20. An implantable medical device, comprising:
a reservoir including a movable member that is movable between a reservoir full position and a reservoir empty position;
a fill port in fluid communication with the reservoir; and
a valve, between the reservoir and the fill port, which includes
a valve housing defining an inner surface,
a valve seat, defining a seal surface and an aperture, associated with the valve housing,
a valve element that is movable between an open position in spaced relation to the seal surface and a closed position in contact with the seal surface, and
a variable length connector that operably connects the valve element to the movable member, the variable length connector having an expandable structure with a first end associated with the valve element, a second end associated with the movable member, and a cap carried by the expandable structure that abuts the inner surface of the valve housing and includes a plurality of apertures.

21. An implantable medical device as claimed in claim 20, wherein the connector cap abuts the valve seat unless the reservoir is at least substantially full.

22. An implantable medical device as claimed in claim 21, wherein
the valve seat includes a plurality of fluid channels associated with the valve seat aperture; and
the plurality of fluid channels and the plurality of cap apertures are respectively sized and positioned such that at least one cap aperture will be at least partially aligned with at least one fluid channel regardless of the orientation of the cap relatively to the valve seat.

23. An implantable medical device as claimed in claim 20, wherein the expandable structure defines a fully extended state and includes a bias element that biases the expandable structure to the fully extended state.

24. An implantable medical device as claimed in claim 20, wherein the expandable structure comprises a telescoping structure with at least three telescoping members that are movable relative to one another.

25. An implantable medical device as claimed in claim 24, wherein
the telescoping structure defines a fully extended state and includes a bias element that biases the telescoping structure to the fully extended state.

26. An implantable medical device as claimed in claim 25, wherein the bias element comprises a spring located outside the telescoping structure.
